(19) United States
(12) Patent Application Publication
(10) Pub. No.: US 2023/0212118 A1
(43) Pub. Date: Jul. 6, 2023

(54) PREPARATION OF AN SELECTIVE ESTROGEN RECEPTOR DEGRADER

(71) Applicant: Recurium IP Holdings, LLC, San Diego, CA (US)

(72) Inventors: Peter Qinhua Huang, San Diego, CA (US); Sayee Gajanan Hegde, San Diego, CA (US); Kevin Duane Bunker, Escondido, CA (US); John Knight, Hereford (GB); Joseph Robert Pinchman, San Diego, CA (US); Aditya Krishnan Unni, San Diego, CA (US); Rakesh Kumar Sit, San Diego, CA (US); Shuguang Zhu, San Diego, CA (US); Chad Daniel Hopkins, San Diego, CA (US); Ian Scott, Niwot, CO (US)

(21) Appl. No.: 17/996,598

(22) PCT Filed: Apr. 21, 2021
(86) PCT No.: PCT/US2021/028344

§ 371 Date: Oct. 19, 2022

Related U.S. Application Data
(60) Provisional application No. 63/013,686, filed on Apr. 22, 2020.

(51) Int. Cl. C07D 209/16; C07D 471/04; B01J 31/12; B01J 23/44; B01J 23/02

(52) U.S. Cl. CPC C07D 209/16; C07D 471/04; B01J 31/122; B01J 23/44; B01J 23/02; C07B 2200/13

(57) ABSTRACT

Described herein is a method for obtaining a selective estrogen receptor degrader, and compounds used in preparing the selective estrogen receptor degrader.

### INCORPORATION BY REFERENCE TO ANY PRIORITY APPLICATIONS

Any and all applications for which a foreign or domestic priority claim is identified, for example, in the Application Data Sheet or Request as filed with the present application, are hereby incorporated by reference under 37 CFR 1.57, and Rules 4.18 and 20.6, including U.S. Provisional Application No. 63/013,686, filed Apr. 22, 2020.

### BACKGROUND

### Field

The present application relates to the fields of chemistry and medicine. More particularly, disclosed herein are method for preparing a compound that can be a selective estrogen degrader that may be used as an anti-cancer agent.

### Description

New methods for preparing chiral compounds with high enantiomeric purity while minimizing undesirable side products are highly valuable. Several chiral compounds can be used as pharmaceutical agents. One class of useful agents are selective estrogen receptor degraders (SERDs) that can be used treat breast cancer.

### SUMMARY

Some embodiments disclosed herein generally related to a compound of Formula (B), and a method of obtaining the same.

Other embodiments disclosed herein generally related to a compound of Formula (F), and a method of obtaining the same.

### BRIEF DESCRIPTION OF THE DRAWINGS

FIG. 1 shows an X-ray powder diffraction pattern of crystalline Compound (C).

### DETAILED DESCRIPTION

### Definitions

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

As used herein, any “R” group(s) such as, without limitation, R¹ represents a substituent that can be attached to the indicated atom(s). Such R groups may be referred to herein in a general way as “R” groups.

As used herein, “Cₐ to C_{b}” in which “a” and “b” are integers refer to the number of carbon atoms in an alkyl group. That is, the alkyl can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated with regard to an alkyl, the broadest range described in these definitions is to be assumed.

As used herein, “alkyl” refers to a straight or branched hydrocarbon chain that comprises a fully saturated (no double or triple bonds) hydrocarbon group. The alkyl group may have 1 to 10 carbon atoms (whenever it appears herein, a numerical range such as “1 to 10” refers to each integer in the given range; e.g., “1 to 10 carbon atoms” means that the alkyl group may consist of 1 carbon atom, 2 carbon atoms, 3 carbon atoms, etc., up to and including 10 carbon atoms, although the present definition also covers the occurrence of the term “alkyl” where no numerical range is designated). The alkyl group could also be a lower alkyl having 1 to 6 carbon atoms. The alkyl group of the compounds may be designated as “C₁-C₄ alkyl” or similar designations. By way of example only, “C₁-C₄ alkyl” indicates that there are one to four carbon atoms in the alkyl chain, i.e., the alkyl chain is selected from methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl and hexyl. The alkyl group may be substituted or unsubstituted.

The term “halide” or “halogen” as used herein, means any one of the radio-stable atoms of column 7 of the Periodic Table of the Elements, such as, fluorine, chlorine, bromine and iodine.

As used herein, the abbreviations for any protective groups, amino acids and other compounds, are, unless indicated otherwise, in accord with their common usage, recognized abbreviations, or the IUPAC-IUB Commission on Biochemical Nomenclature (See, Biochem. 11:942-944 (1972)).

The term “pharmaceutically acceptable salt” refers to a salt of a compound that does not cause significant irritation to an organism to which it is administered and does not abrogate the biological activity and properties of the compound. In some embodiments, the salt is an acid addition salt of the compound. Pharmaceutical salts can be obtained by reacting a compound with inorganic acids such as hydrohalic acid (e.g., hydrochloric acid or hydrobromic acid), sulfuric acid, nitric acid and phosphoric acid. Pharmaceutical salts can also be obtained by reacting a compound with an organic acid such as aliphatic or aromatic carboxylic or sulfonic acids, for example formic, acetic, succinic, lactic, malic, tartaric, citric, ascorbic, nicotinic, methanesulfonic, ethanesulfonic, p-toluenesulfonic, salicylic or naphthalenesulfonic acid. Pharmaceutical salts can also be obtained by reacting a compound with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, C₁-C₇ alkylamine, cyclohexylamine, triethanolamine, ethylenediamine, and salts with amino acids such as arginine and lysine.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; and use of terms like ‘preferably,’ ‘preferred,’ ‘desired,’ or ‘desirable,’ and words of similar meaning should not be understood as implying that certain features are critical, essential, or even important to the structure or function, but instead as merely intended to highlight alternative or additional features that may or may not be utilized in a particular embodiment. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

It is understood that, in any compound described herein having one or more chiral centers, if an absolute stereochemistry is not expressly indicated, then each center may independently be of R-configuration or S-configuration or a mixture thereof. Thus, the compounds provided herein may be enantiomerically pure, enantiomerically enriched, racemic mixture, diastereomerically pure, diastereomerically enriched, or a stereoisomeric mixture. In addition it is understood that, in any compound described herein having one or more double bond(s) generating geometrical isomers that can be defined as E or Z, each double bond may independently be E or Z, or a mixture thereof.

Likewise, it is understood that, in any compound described, all tautomeric forms are also intended to be included.

It is to be understood that where compounds disclosed herein have unfilled valencies, then the valencies are to be filled with hydrogens or isotopes thereof, e.g., hydrogen-1 (protium) and hydrogen-2 (deuterium).

It is understood that the compounds described herein can be labeled isotopically. Substitution with isotopes such as deuterium may afford certain therapeutic advantages resulting from greater metabolic stability, such as, for example, increased in vivo half-life or reduced dosage requirements. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Where a range of values is provided, it is understood that the upper and lower limit, and each intervening value between the upper and lower limit of the range is encompassed within the embodiments.

Some embodiments disclosed herein generally related to a compound of Formula (B), and a method of obtaining the same, wherein a compound of Formula (B) has the structure

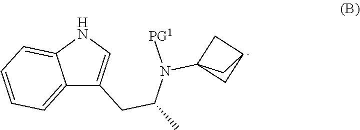

Other embodiments disclosed herein generally related to a compound of Formula (F), and a method of obtaining the same, wherein a compound of Formula (F) has the structure

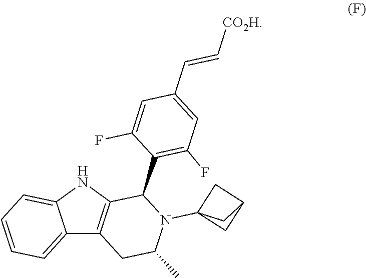 [SMILES: C[C@@H]1Cc2c([nH]c3ccccc23)[C@@H](c2c(F)cc(/C=C/C(=O)O)cc2F)N1C12CC(C1)C2]

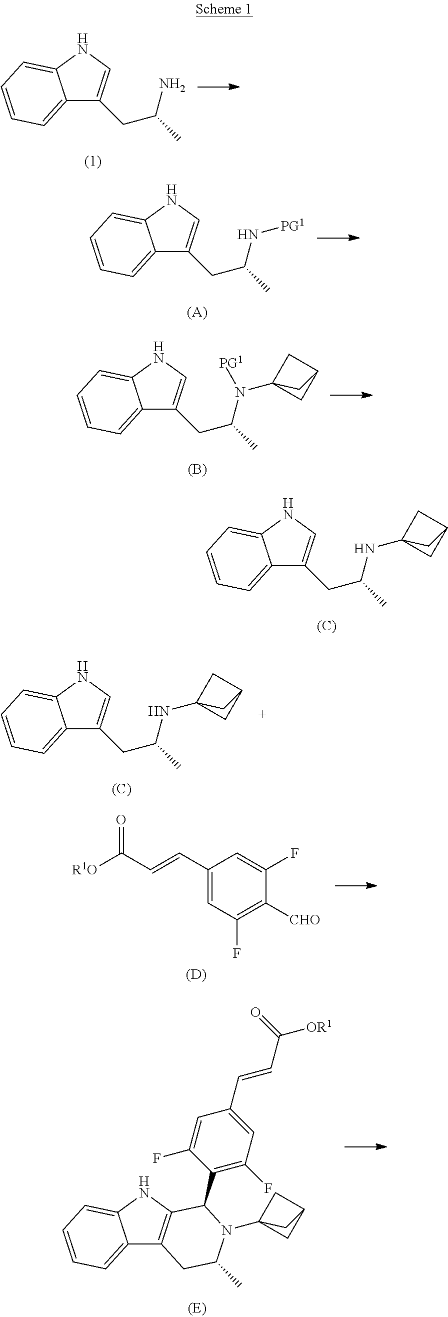

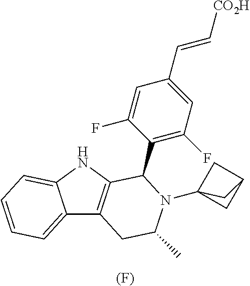

As shown in Scheme 1, a compound of Formula (1) can be reductively aminated using an aldehyde and a reducing agent to provide a compound of Formula (A).

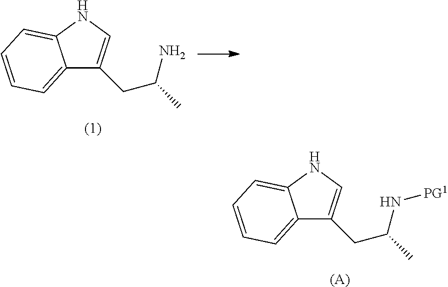

A variety of reducing agent can be used for the reductive amination of a compound of Formula (1). Examples of appropriate reducing agents include sodium borohydride, lithium aluminum hydride, sodium triacetoxyborohydride and sodium cyanoborohydride. Similarly, a variety of aldehydes can be used to provide a compound of Formula (A). Exemplary aldehydes are an unsubstituted or a substituted benzylaldehyde or an unsubstituted or a substituted C₁₋₆ alkylaldehyde. In some embodiments, the aldehyde can be an unsubstituted or a substituted benzylaldehyde.

A compound of Formula (B) can be obtained by combining a compound of Formula (A), a base and [1.1.1]propellane to afford a compound of Formula (B), wherein each PG¹ can be a protecting group.

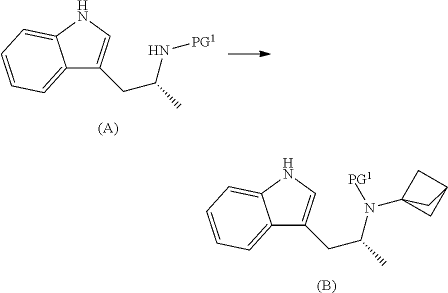

Various protecting groups can be used for each PG¹. Examples of suitable protecting groups include an unsubstituted or a substituted benzyl, a silyl-based protecting group and an unsubstituted allyl. In some embodiments, each PG¹ can be an unsubstituted or a substituted benzyl. In some embodiments, each PG¹ can be an unsubstituted benzyl.

A variety of bases can be used to obtain a compound of Formula (B) from a compound of Formula (A). In some embodiments, the base can be organometallic base. Suitable organometallic bases are known to those skilled in the art. Two examples of suitable organometallic bases are an organometallic magnesium base (for example a Grignard reagent) or an organometallic lithium base (such as n-butyllithium). Another example of a suitable organometallic base is an organometallic magnesium-lithium base. In some embodiments, the organometallic magnesium-lithium base can have the formula (unsubstituted C₁₋₄ alkyl)Mg(halide)-Li(halide), such as iPrMgCl·LiCl.

The PG¹ from a compound of Formula (B) can be removed to obtain a compound of Formula (C).

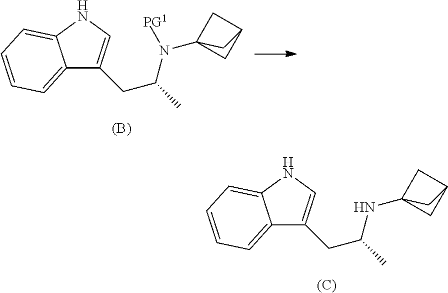

One method for removing the PG¹ of the compound of Formula (B) is via metal catalyzed hydrogenation. Exemplary metal catalyzed hydrogenation can be palladium catalyzed hydrogenation, platinum catalyzed hydrogenation and nickel catalyzed hydrogenation. Various catalysts can be used for metal catalyzed hydrogenation, and include catalysts selected from Pd(OH)₂, Pd/C, Pd(OH)₂/C, silica supported Pd, resin supported Pd, polymer supported Pd, Raney nickel, Urushibara nickel, Ni supported on SiO₂, Ni supported on TiO₂—SiO₂, Pt/C, Pt supported on SiO₂ and Pt supported on TiO₂—SiO₂. In some embodiments, the PG¹ of a compound of Formula (B) can be removed using H₂ and a Pd compound. Another method for removing the PG¹ of a compound of Formula (B) is by using a fluoride source or an acid. A variety of fluoride sources can be used. Examples of fluoride sources include pyridine hydrogen fluoride complex, a triethylamine hydrogen fluoride complex, NaF, tetrabutylammonium fluoride (TBAF) and 1:1 tetrabutylammonium fluoride/AcOH.

A compound of Formula (C) and a compound of Formula (D), optionally in the presence of an acid, can be combined to form a compound of Formula (E).

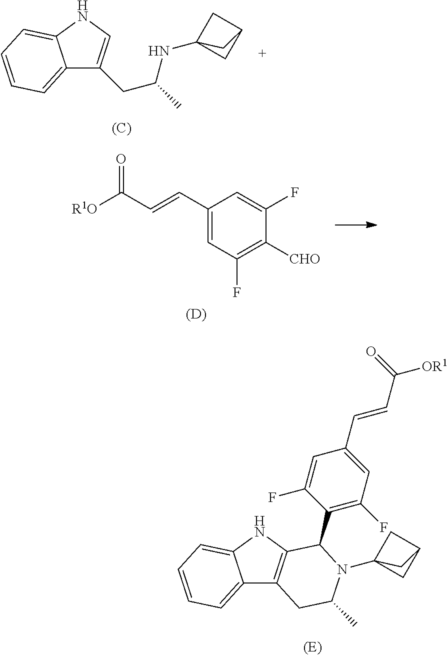

wherein each R¹ is an unsubstituted C₁₋₄ alkyl.

Several acids can be used to form a compound of Formula (E) from a compound of Formula (C) and a compound of Formula (D). In some embodiments, the acid can be an acetic acid. Those skilled in the art understand that a compound of Formula (C) and a compound of Formula (D) can undergo a condensation reaction between the secondary amine of the compound of Formula (C) and the aldehyde of the compound of Formula (D), and then a cyclization reaction to form a compound of Formula (E). In some embodiments, R¹ of a compound of Formula (D) and a compound of Formula (E) can be methyl.

Hydrolyzing the alkyl ester (—C(═O)OR¹, wherein R¹ is an unsubstituted C₁₋₄ alkyl) of the compound of Formula (E) to a carboxylic acid and afford a compound of Formula (F).

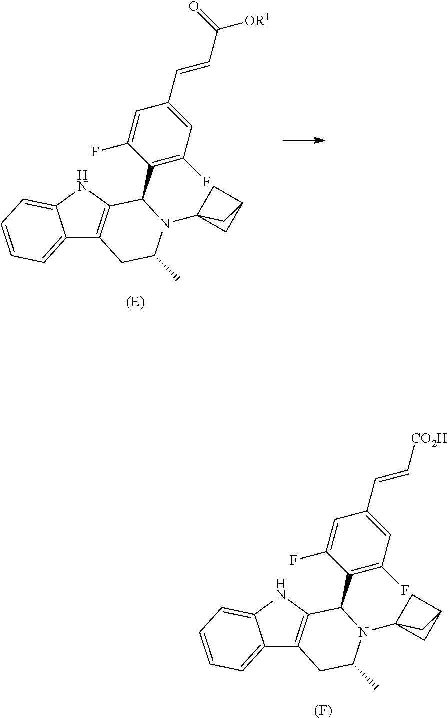

In some embodiments, the hydrolysis can be conducted using a base. Various bases can be used, and include NaOH, LiOH and KOH. In some embodiments, R¹ of a compound of Formula (E) can be methyl.

The hydrogen sulfate salt of the compound of Formula (F) can be obtained through the use of an appropriate hydrogen sulfate source. An exemplary hydrogen sulfate source is H₂SO₄.

Several compounds provided herein include [1.1.1]propellane. In some embodiments, [1.1.1]propellane can be obtained from dibromo-2,2-bis(chloromethyl)cyclopropane using Mg(0) or an organolithium reagent. Those skilled in the art know appropriate organolithium reagents, such as PhLi and (C₁₋₈ alkyl)Li.

There are several advantages of the synthesis shown in Scheme 1. A non-limiting list of advantages includes increased yield(s) compared to previous known synthesis, none to little need for column purification (such as achiral or chiral purification with silica gel, HPLC or SFC), minimal loss of materials (for example, the synthesis in Scheme 1 uses chirally pure or chirally enriched starting material(s) compared to the same procedure that uses racemic starting material(s)), less purification steps, high chiral purity of compound(s) (such as those shown in a synthetic scheme provided herein), improved and/or more reliable impurity control(s) and/or procedures optimized for the manufacture of compounds described herein on kilogram to greater than kilogram scale.

With respect to obtaining [1.1.1]propellane as described herein, there are also several advantages compared to those procedures known in the art. Examples of advantages include cost effectiveness due to the chosen starting materials (for example, due to the cost of magnesium), a more simple procedure (for example, a procedure described herein is more simple because of the temperature used in the reaction (e.g., >0° C. or >25° C.), there is little to no need for the use of a cryogenic chiller when magnesium is used to make [1.1.1]propellane and/or the ease to recover unreacted starting materials), a more clean reaction (for example, due to less waste and/or the reduced need for organometallic reagents when magnesium is used to make propellane) and/or ability to scale-up the reaction (for example, to >10 kg scale, >20 kg scale or >30 kg scale).

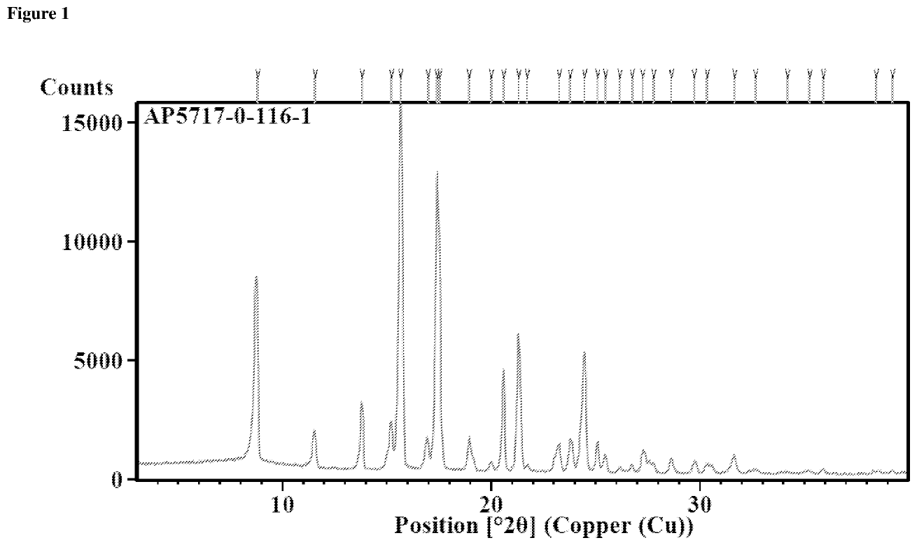

Further advantages of a procedure described herein can be the preparation of a compound described herein in a crystalline form. For example, Compound (C) can be obtained in a crystalline form. An X-ray powder diffraction pattern of crystalline Compound (C) is provided in FIG. 1, and the peaks, °2θ, d-spacing [Λ] and Relative Intensity [%] is provided in Table 1.

TABLE 1

| | | | Relative |
|---|---|---|---|
| Peak | °2θ | d-spacing [Å] | Intensity [%] |
| 1 | 8.8 | 10.06 | 44.16 |
| 2 | 11.6 | 7.66 | 9.76 |
| 3 | 13.8 | 6.41 | 17.93 |
| 4 | 15.2 | 5.83 | 13.77 |
| 5 | 15.6 | 5.67 | 100.00 |
| 6 | 16.9 | 5.23 | 8.64 |
| 7 | 17.4 | 5.10 | 83.12 |
| 8 | 17.5 | 5.06 | 63.27 |
| 9 | 18.9 | 4.69 | 9.43 |
| 10 | 20.00 | 4.44 | 2.37 |
| 11 | 20.6 | 4.31 | 27.98 |
| 12 | 21.3 | 4.17 | 38.94 |
| 13 | 21.7 | 4.09 | 1.80 |
| 14 | 23.3 | 3.82 | 7.83 |
| 15 | 23.8 | 3.74 | 8.54 |
| 16 | 24.5 | 3.64 | 33.57 |
| 17 | 25.1 | 3.55 | 8.56 |
| 18 | 25.5 | 3.50 | 4.53 |
| 19 | 26.2 | 3.41 | 1.46 |
| 20 | 26.7 | 3.33 | 2.15 |
| 21 | 27.2 | 3.27 | 5.90 |
| 22 | 27.8 | 3.21 | 2.80 |
| 23 | 28.6 | 3.12 | 4.09 |
| 24 | 29.7 | 3.01 | 3.36 |
| 25 | 30.4 | 2.94 | 2.79 |
| 26 | 31.6 | 2.83 | 5.23 |
| 27 | 32.7 | 2.74 | 1.15 |
| 28 | 34.2 | 2.62 | 0.47 |
| 29 | 35.3 | 2.55 | 0.79 |
| 30 | 35.9 | 2.50 | 1.28 |
| 31 | 38.4 | 2.34 | 0.65 |
| 32 | 39.2 | 2.30 | 0.74 |

In some embodiments, crystalline Compound (C) can be characterized by one or more peaks in an X-ray powder diffraction pattern, wherein the one or more peaks can be range of from 8.0 to 9.6 °2θ, from 14.8 to 16.4 °2θ, from 16.6 to 18.3 °2θ, from 19.8 to 21.4 °2θ, from 20.5 to 22.1 °2θ and from 23.7 to 25.3 °2θ. In some embodiments, crystalline Compound (C) can be characterized by one or more peaks in an X-ray powder diffraction pattern, wherein the one or more peaks can be selected from 8.8 °2θ±0.2 °2θ, 15.6 °2θ±0.2 °2θ and 17.4 °2θ±0.2 °2θ. In some embodiment, crystalline Compound (C) can be characterized by one or more peaks in an X-ray powder diffraction pattern, wherein the one or more peaks can be selected from 20.6 °2θ±0.2 °2θ, 21.3 °2θ±0.2 °2θ and 24.5 °2θ±0.2 °2θ. In some embodiments, crystalline Compound (C) can exhibit an X-ray powder diffraction pattern as shown in FIG. 1. All XRPD patterns provided herein are measured on a degrees 2-Theta (2θ) scale. It should be understood that the numerical values of the peaks of an X-ray powder diffraction pattern may vary from one machine to another, or from one sample to another, and so the values quoted are not to be construed as absolute, but with an allowable variability, such as ±0.2 degrees two theta (2θ), or more. For example, in some embodiments, the value of an XRPD peak position may vary by up to ±0.2 degrees 2θ while still describing the particular XRPD peak.

### EXAMPLES

Additional embodiments are disclosed in further detail in the following examples, which are not in any way intended to limit the scope of the claims.

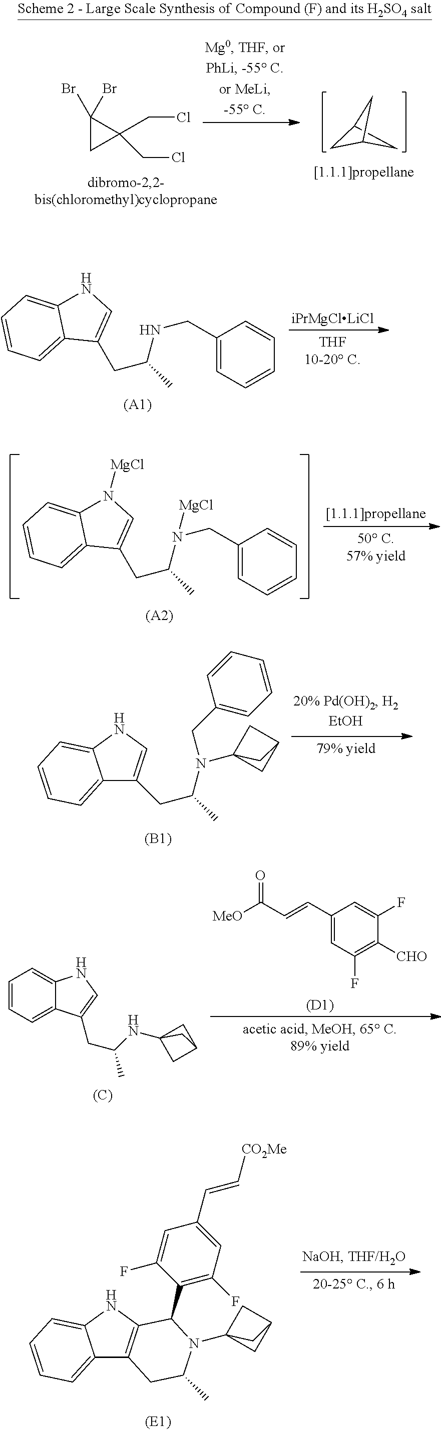

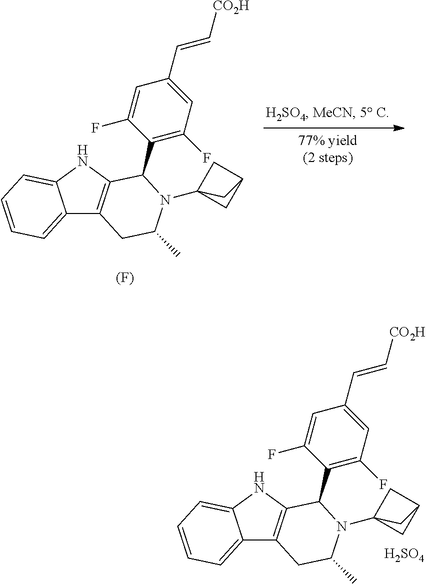

### Example 1: Synthesis of Compound (B1)

To a dried three-necked flask (20 L), equipped with thermometer and mechanical stirrer, was charged dibromo-2,2-bis(chloromethyl)cyclopropane (1.60 kg, 5.39 mol, 1.0 eq.) and n-Bu₂O. The mixture was cooled to −60±5° C. (Tᵢₙ) in a dry ice-EtOH bath, and a yellow suspension was formed. To the mixture was added dropwise a solution of PhLi (1.44 M, 6.25 kg, 10.8 mol, 2.0 eq.) in n-Bu₂O via a dropping funnel over 3 h at −60±5° C. (Tᵢₙ). The mixture was stirred at −60±5° C. (Tᵢₙ) for 1 h. The mixture was then warmed up to 0±5 (Tᵢₙ) over 0.5 h and then stirred at 0±5° C. (Tᵢₙ) for 2 h in an ice-water bath. In parallel, to a 100 L glass reactor was added a solution of i-PrMgCl·LiCl (1.05 M, 47.8 kg, 50.53 mol, 1.875 eq.) in THF. The mixture was cooled to at 10±5° C. (Tᵢₙ). To this mixture was added a solution of Compound (A1) (8.91 kg, 33.7 mol, 1.25 eq.) in THF (28.5 kg, 4.0 v) slowly over a 1 h period at 10-25° C. The mixture was stirred at 20±5° C. (Tᵢₙ) for an additional 1 h after the addition was complete. To a 200 L reactor was transferred the [1.1.1]propellane solution followed by the dianion of Compound (A1) with vigorous stirring. The reactor was sealed and warmed to 35-40° C., 40-45° C. and 45-50° C. in sequence. Each stage was maintained for 5 h. The reaction was then transferred into cooled (0±5° C.) aq. ammonium chloride solution with vigorous stirring. The phases were separated, and the aqueous phase was extracted with EtOAc. The organic phases were combined, washed with sat. aq. NaCl solution and then dried over anhydrous Na₂SO₄ for more than 1 h. The mixture was filtered and washed with EtOAc. The filtrate was concentrated under reduced pressure to remove the majority of THF and EtOAc. The residue was transferred into a 100 L glass reactor and neat formic acid (1.05 eq. relative to the amount of unreacted Compound (A1) was added to the mixture at 25° C. After 3 h, the suspended solid was filtered. The mother liquor was concentrated at 45-50° C. to remove the majority of n-Bu₂O and provided Compound (B1) (12.1 kg) that was dissolved in 10 v of n-heptane and purified by column chromatography through 4 w/w of 60-100 mesh silica gel eluting with a EtOAc/n-heptane gradient. Concentration of fractions afforded Compound (B1) (5.1 kg, 57% yield). ¹H NMR (300 MHz, CDCl₃-d) δ 7.85-7.97 (br s, 1H), 7.53 (d, J=7.9 Hz, 1H), 7.36 (app t, J=7.5 Hz, 3H), 7.32-7.14 (m, 4H), 7.09 (t, J=7.5 Hz, 1H), 7.00 (d, J=2.0 Hz, 1H), 3.87 (d, J=15.1 Hz, 1H), 3.76 (d, J=15.1 Hz, 1H), 3.59-3.33 (m, 1H), 3.09 (dd, J=14.1, 4.8 Hz, 1H), 2.71 (dd, J=14.1, 9.6 Hz, 1H), 2.30 (s, 1H), 1.92-1.70 (m, 6H), 1.06 (d, J=6.6 Hz, 3H). MS (ESI) m/z 331.1 [M+H]⁺.

### Example 2: Synthesis of Compound (C)

A solution of Compound (B1) (1.67 kg, 5.05 mol, 1.0 eq.) in EtOH (8.4 L, 5 v) was evacuated and refilled with N₂ (3×). 20% Pd(OH)₂/C (200 g, 12 wt % loading) was charged into the flask. The system was evacuated and refilled with N₂ (3×), followed by evacuation and refilling with H₂ (3×). The mixture was stirred at 25-30° C. for 16 h under 1 atm of H₂. The mixture was filtered through a pad of diatomaceous earth under an atmosphere of N₂. The catalyst/diatomaceous earth pad was washed with EtOH (2×2v) under an atmosphere of N₂. The filtrate was concentrated. The resulting oily product was dissolved in EtOAc (˜1v×2) and concentrated under reduced pressure at 45° C. (2×). The oily product was dissolved in n-heptane (2v) and concentrated under reduced pressure at 45° C. The resulting oil was slurried in n-heptane (1290 mL, 3v) with stirring overnight. The slurry was filtered, and the filter cake was dried to constant weight under reduced pressure at 45° C. The above procedure was repeated 4 more times in total (3×1.67 kg+0.9 kg lots of starting material). The batches were pooled to afford Compound (C) (3.4 kg, 79% yield). ¹H NMR (300 MHz, CDCl₃-d) δ 8.16-7.95 (br s, 1H), 7.62 (d, J=7.9 Hz, 1H), 7.36 (d, J=8.0 Hz, 1H), 7.19 (t, J=7.4 Hz, 1H), 7.12 (t, J=7.4 Hz, 1H), 7.02 (s, 1H), 3.25-3.11 (m, 1H), 2.88 (dd, J=14.2, 7.2 Hz, 1H), 2.74 (dd, J=14.2, 6.5 Hz, 1H), 2.36 (s, 1H), 1.95-1.51 (m, 6H), 1.12 (d, J=6.2 Hz, 3H). MS (ESI) m/z 240.9 [M+H]⁺.

### Example 3: Synthesis of Compound (E1)

To a 80 L glass reactor was added methanol (11.7 kg) and acetic acid (3.3 kg). Compound (D1) ((E)-methyl3-(3,5-difluoro-4-formyphenyl) acrylate) (6.9 kg) was added into the mixture through a solid addition funnel. The solid addition funnel was rinsed with methanol (2.7 kg) and was added to the reactor. The mixture was heated to 60-70° C. at a reference rate of 5-15° C./h. Into a separate drum, Compound (C) (6.6 kg) was added through a solid addition funnel, and MeOH (4.2 kg) was used to rinse the additional funnel The solution of Compound (C) was added into the reactor at a reference rate of 6-12 kg/h at 60-70° C. The mixture was allowed to react at 60-70° C. for 14-16 h. The mixture was then cooled to 15-25° C. at a reference rate of 10-20° C./h. The mixture was maintained and stirred for 2-3 h. The mixture was filtered with a 20 L Nutsche filter. The filter cake was washed with additional methanol before drying at T≤40° C. to afford Compound (E1) (10.9 kg, 88.9% yield). ¹H NMR (300 MHz, DMSO-d₆) δ 10.48 (br s, 1H), 7.63 (d, J=18.0 Hz, 1H), 7.50 (d, J=10.2 Hz, 2H), 7.38 (d, J=6.9 Hz, 1H), 7.17 (d, J=7.2 Hz, 1H), 7.01-6.91 (m, 2H), 6.80 (d, J=16.2 Hz, 1H), 5.33 (s, 1H), 3.73 (s, 3H), 3.61 (br s, 1H), 3.01-2.93 (m, 1H), 2.57 (d, J=16.2 Hz, 1H), 2.24 (s, 1H), 1.77 (d, J=9.0 Hz, 3H), 1.57 (d, J=9.0 Hz, 3H), 1.08 (d, J=6 Hz, 3H). MS (ESI) m/z 449.10 [M+H]⁺.

### Example 4: Synthesis of Compound F and its H₂SO₄ Salt

THF (13.3 kg) was added into a 80 L reactor at 15˜25° C. followed by Compound (E1) (7.5 kg) at 15˜25° C. At 15˜25° C., the solution of NaOH (1.0 kg) in purified water (30.0 kg) was added into the mixture at a rate of 10˜15 kg/h. The mixture was allowed to react at 15˜25° C. After 18˜20 h, the mixture was transferred into the 200 L glass-lined reactor. The mixture was then concentrated at T≤40° C. under reduced pressure until 3.3˜4.0V left. Purified water (7.5 kg) was added into the mixture at T≤40° C. The mixture was then concentrated at T≤40° C. under reduced pressure (P≤−0.08 MPa) until 3.3˜4.0V left. The mixture was cooled to 5˜15° C. at a reference rate of 10˜15° C./h. At T≤15° C., the mixture was adjusted pH to 7.5˜8.0 with a solution of sulfuric acid (1.5 kg) in purified water (29.9 kg). Ethyl acetate (23.6 kg) was added, and the mixture was stirred for 10˜30 min until the solids dissolve completely by visual check. The temperature of the mixture was adjusted to 5˜15° C. At T≤15° C., the mixture was adjusted pH to 6.0-6.3 with a sulfuric acid solution. At T≤15° C., the mixture was adjusted to a pH of 5.1˜5.4 with a solution of sulfuric acid (0.4 kg) in purified water (15.0 kg). The mixture was stirred for 15˜30 min at T≤15° C. and then settled for 0.5˜1 h before separation. The aqueous phase was extracted with ethyl acetate (total of ˜50 kg) (2×) at T≤15° C. The mixture was stirred for 15˜30 min and settled for 0.5˜1 h before separation. The mixture in the 80 L glass reactor was concentrated at T≤40° C. under reduced pressure until 14˜16 L left. THF (total of 50 kg) was added into the reactor followed by repeated concentration four times. The mixture was finally concentrated at T≤40° C. under reduced pressure until 14˜16 L left. THF (13.4 kg) was added into the mixture, and the mixture was transferred into 200 L hastelloy reactor. THF (5.7 kg) was added followed by purified water (1.9 kg). The mixture was cooled to 5˜15° C., and a solution of sulfuric acid (1.7 kg) in acetonitrile (28.7 kg) was added at a reference rate of 5˜15 kg/h. The temperature was adjusted to 15˜25° C. and maintained for 3˜5 h under stirring. The mixture was filtered with a 220 L hastelloy alloy agitating filter dryer followed by additional rinsing with acetonitrile. The solid was dried at T≤40° C. to afford Compound (F) as a H₂SO₄ salt (6.9 kg, 76.9% yield) with a purity>99%. ¹H NMR (400 MHz, CD₃OD) δ 7.65 (d, J=16.0 Hz, 1H), 7.54 (d, J=7.9 Hz, 1H), 7.48 (d, J=10.4 Hz, 2H), 7.31 (d, J=8.2 Hz, 1H), 7.19-7.14 (m, 1H), 7.12-7.07 (m, 1H), 6.67 (d, J=16.0 Hz, 1H), 6.18 (s, 1H), 4.39-4.26 (m, 1H), 3.53-3.40 (m, 1H), 3.19-2.99 (m, 1H), 2.69 (br s, 1H), 2.38-1.97 (m, 6H), 1.64 (d, J=6.8 Hz, 3H). MS (ESI) m/z 435.13 [M+H]⁺.

### Example 5: Large Scale Production of Compound (C) Using MeLi Generated [1.1.1]Propellane

To the reactor was added MeLi (321.10 kg, 2.0 M in DEM), cooled to −50-−65° C., followed by the addition of dibromo-2,2-bis(chloromethyl)cyclopropane ((99.74 kg, 1.0 eq.) as a solution in DEM (2.0 V) dropwise keeping internal temperature between −50-−65° C. The mixture was stirred for at least 4 h and then allowed to warm to −30±5° C. over at least 3.0 h. The mixture was warmed to 0±5° C. over at least 3 h to ensure starting material was consumed. The mixture was cooled to −5° C. and then N-methylpiperazine (84.25 kg, 2.5 eq.) in DEM (1.0 V) was added. The mixture was allowed to warm to 10±5° C. and stirred over approximately 12 h. The mixture was filtered and then distillation was done ensuring that the inner temperature of the reactor rose to no higher than 28° C. (the vacuum≤−0.095 MPa) while the receiving vessel was cooled to −55±5° C. The distillate was warmed to 15±5° C., and CH₃SO₃H (3.90 kg, 1.2 eq.) was added to quench the residual N-methylpiperazine in the distillate. The mixture was stirred for at least 2 hours. Once more the distillation was complete to afford a solution of [1.1.1]propellane in DEM.

To a separate reactor was added i-PrMgCl·LiCl in THF solution (1.3 M) (211.70 kg, 2.2 eq.) to the reactor at 25±5° C. The mixture was cooled and Compound (A1) was added as a solution in THF (33.6% wt/wt, 1.0 eq., 31 kg Compound (A1)) while maintaining the temperature at 20±10° C. for at least 1 h. The above [1.1.1]propellane (1.5% assay, 1.0 eq.) was added over at least 2 h. The mixture was heated at 50±5° C. After 15 h, the mixture was cooled. 15% wt/wt ammonium chloride (382.4 kg, 10.0V) was added over 3 h and then warmed to 25 C for at least 1 hour. The mixture was separated, and the organic phase was washed with softened water (5V×2). The separated organic phase was concentrated to 2-3V in-vacuo with an external temperature no higher than 45° C. A solvent swap with MTBE (3×5V) was done to remove DEM and THF below pre-specified levels. The residual starting material (Compound (A1)) was removed by adding the appropriate amount of formic acid (1.05 eq. based on calculated Compound (A1)) in MTBE over 1 h following by stirring for at least 1 h at 20° C. The formate salt of Compound (A1) was filtered. The filtrate was washed with softened water and concentrated in-vacuo to 2-3V followed by a solvent swap with dichloromethane (228.10 kg, 5V). Added silica gel (100-200 mesh) (78.60 Kg, −2.5×wt of Compound (A1) that was initiated used), quartz sand (5.03 kg) followed by heptane (176.55 Kg, 8V), and the mixture was filtered through a Nutsche filter. Dichloromethane was used to wash the pad. The combined filtrates were then subjected to a microporous filter. The organic phase was concentrated to 2-3V in-vacuo with the inner temperature maintained to no higher than 40° C. Solvent swap with EtOH afforded a solution of the crude mixture in EtOH (yield: 39% based on assay of 9.7% w/w; HPLC purity: 97.6%)

The crude ethanolic mixture of Compound (B1) (163.45 kg, 1.0 eq., assay: 9.7% w/w) was charged to the reactor followed by softened water (5.05 kg, 3% wt) and citric acid (0.206 kg, 0.02 eq.) under N₂ flow. Palladium hydroxide (1.90 kg, 12% wt/wt) was added. Hydrogen was charged to the autoclave to pressure of 0.5±0.2 MPa, and the autoclave was then slowly heated to 20±10° C. The reaction was stopped after 36 h based on specifications and filtered. The cake was washed with additional EtOH ((75.70 kg, 6V). The filtrate was concentrated in-vacuo controlling the inner temperature at no higher than 40° C. to about 20 L. Solvent swap with heptane (75 L, 5V) was done twice in-vacuo. The mixture was then heated to 75±5° C., and the particulates were removed. The mixture was cooled and then filtered affording Compound (C) (2.15 kg product, 99.6% purity). The residual material was dissolved in MTBE (50 L) and treated with 10% ammonia in water (30 L). The organic phase was then subjected to a short pad of silica gel (14 kg, 0.88 wt/wt relative to original amount of Compound (A1)) followed by the additional MTBE (55 L). After combining the filtrates and the previously obtained Compound (C) (2.15 kg), a solvent swap with heptane (75 L, 5V) was done twice. The mixture was cooled to 5±5° C. after 1 h to provide Compound (C) (9.07 kg, 78% yield, 98.2% HPLC purity)

### Example 6: Preparation of Propellane Solution

Magnesium turnings (7.29 grams (300 mmol) were added to an oven dried 500 mL single neck flask containing a stirbar. The flask was fitted with a rubber septum with a digital thermocouple such that the tip of the thermocouple was at the base of the flask. The flask was evacuated and backfilled with N₂ while still hot. After reaching room temperature, anhydrous THF (50 mL) was added followed by the dropwise addition of a 1.0M solution of diisobutylaluminum hydride in THF (10 mL). The magnesium turnings were stirred in the flask for 1-3 h to fully activate the turnings. After the 1-3 h, an additional THF (30 mL) was added to the flask containing then Mg turnings, which was immersed in a water bath maintained at room temperature to moderate the reaction temperature. A solution of dibromo-2,2-bis(chloromethyl)cyclopropane (30 g dissolved in THF (90 mL)) was added to the solution of magnesium turnings via cannula dropwise over 60 min, ensuring the temperature of the solution stayed between 20 and 35° C. After the addition was complete, the reaction was stirred for an additional 1 h at ambient temperature. To precipitate out most of the magnesium salts, MTBE (100 mL) was added to the reaction which was stirred briefly, and allowed to stand for an additional 30 min. The crude material was then filtered through a small pad of Celite using positive N₂ gas pressure into a separate 250 mL flask. The light brown filtrate was capped with a septum and stored at −20° C. The propellane content in the THF was determined using q-NMR and indicated a yield of 55%. The crude or distilled propellane solutions were used in the synthesis of Compound (F).

### Example 7: [1.1.1]Propellane Synthesized with Magnesium Reacting with Compound (A1) (TMP Present as Additive)

An oven dried 350 mL pressure vessel was cooled with a nitrogen-filled balloon and charged with Compound (A1) (5 g, 18.9 mmol) and THF (37 mL). Isopropylmagnesium chloride lithium chloride (30.3 mL, 37.8 mmol) was added dropwise by controlling the internal temperature at 30-32° C. The mixture was allowed to stir for 2 h at rt. Distilled 2,2,6,6-tetramethylpiperidine (TMP) (6.44 mL, 37.8 mmol) followed by a [1.1.1] propellane solution (1.1 eq., 0.46M in THF, 45.2 mL, 20.8 mmol) was added dropwise. The reaction vessel was sealed and heated at 68° C. for 20 h. NMR indicated 87% conversion into product. The mixture was cooled to 0° C., and H₂O (160 mL) was added followed by EtOAc (160 mL). The organic layers were separated, and the aqueous fraction was extracted with additional EtOAc (100 mL). The combined organic fractions were washed with 15% ammonium chloride solution (60 mL). The organic layer was further washed with a 5% citric acid aqueous solution (3×150 mL), dried over Na₂SO₄ and concentrated in-vacuo to obtain crude Compound (B1) (5 g, 15 mmol, 80% yield).

### Example 8: [1.1.1]Propellane Synthesized with Magnesium Reacting with Compound (A1) (No TMP Present as Additive)

An oven dried 350 mL pressure vessel was cooled with a nitrogen-filled balloon and charged with Compound (A1) (5 g, 18.9 mmol) and THF (37 mL). Isopropylmagnesium chloride lithium chloride (30.3 mL, 37.8 mmol) was added dropwise by controlling the internal temperature at 30-30° C. The reaction was allowed to stir for 2 h at rt. A solution of [1.1.1]propellane (1.1 eq., 0.42 M in THF, 49.5 ml, 20.8 mmol) was added dropwise. The reaction vessel was sealed and heated at 68° C. for 20 h. NMR indicated 70% conversion into product. The mixture was cooled to 0° C., and H₂O (160 mL) was added followed by EtOAc (160 mL). The organic layers were separated, and the aqueous fraction was extracted with additional EtOAc (100 mL). The combined organic fractions were washed with 15% ammonium chloride solution (60 mL). The organic layer was further washed with a 5% citric acid aqueous solution (3×200 mL), dried over Na₂SO₄ and concentrated in-vacuo to obtain crude Compound (B1) (3.6 g, 10.9 mmol, 58% yield).

### Example 9: Large Scale Production of Compound (C) Using Mg-Generated [1.1.1]Propellane

To a 500 L reactor was added dichloromethane (272.0 kg) followed by the HCl salt of Compound (A1) (41.0 kg). The mixture was cooled to 10-25° C. and aqueous NaOH (142.9 kg, 7.9%). The mixture was stirred for 2 h at 5-15° C. The organic phase was extracted with dichloromethane (189 kg). The pooled organic phase was dried with aq. NaCl (22.5% wt/wt, 74 kg) and then dried over sodium sulfate (19.0 kg). The salts were filtered, and the filtrate was concentrated in-vacuo. THF (132 kg) was charged, and solvent was removed in-vacuo. THF was recharged to afford a solution in THF (assay: 35.64% representing 35 kg total of Compound (A1)). Two batches were run to make Compound (B1). Mg turnings (5.8 kg, 238.6 mol) were added to a dried 500-L reactor and THF (132 kg) was added followed by Dibal-H (5.0 kg, 1.0 M in hexane). The mixture was stirred at 20±5° C. for 1 h. The mixture was warmed to 30-35° C. A portion (˜5% of total volume) of a solution of dibromo-2,2-bis(chloromethyl)cyclopropane (29.5 kg dissolved in THF (78 kg)) was added, and then the remaining portion was added over 6 h. The mixture was heated to 40-45° C. for 2 h. A solution of Compound (A1) (17.5 kg based on assay) was added at 0-10° C. over 50 min, followed by the addition of i-PrMgCl·LiCl (106.0 kg, 1.25 M in THF) at 5-15° C. over 2.5 h. The reactor was sealed and warmed to 55° C. for 18 h. The mixture was cooled to 0-5° C. The flask was charged with 5% aqueous citric acid (20 kg) at 0-10° C. to quench the reaction. After transferring contents to a 1000 L reactor, additional 5% citric acid solution (220 kg) was added at 0-10° C. to adjust pH to 7˜8. After 2 h, the layers were separated, and the aqueous layer was extracted with MTBE (2×160 kg). A second batch on same scale was run and processed in a similar manner. The pooled organic extractions from both batches were washed with 5% aqueous sodium bicarbonate (350 kg). The solution was concentrated at 35˜40° C. under vacuum to 400˜500 L and diluted with MTBE (324 kg). The solution was washed with 5% citric acid, followed by water and 5% aqueous sodium bicarbonate solution. The organic layer was dried over sodium sulfate and filtered. The cake was washed with dichloromethane (50 kg). The filtrate was concentrated at 35˜40° C. under vacuum, and heptane (136 kg) and dichloromethane (50 kg) were charged. The mixture was again concentrated, and the residue was diluted with dichloromethane and petroleum ether. The solution was passed through a pad of silica gel (60-100 mesh). The filtrate was concentrated 40° C., and the residue was dissolved with THF (130 kg). The yield corrected by purity is 35% for this step to make Compound (B1). The solution was charged to a 500 L reactor, and the system was purged with nitrogen (3×). 20% wet Pd(OH)₂/C (2.5 kg) was added, and the system was purged with nitrogen (3×) followed by purging with hydrogen (3×). The slurry was agitated at 25-30° C. under 0.06˜0.08 Mpa H₂ for 40 h. The mixture was filtered through a pad of Celite, and the cake was washed with dichloromethane (100 kg). The filtrate was concentrated under vacuum at 35-40° C. to ˜25 L, and additional dichloromethane was added. The solution was cooled to 5-15° C. 4 M HCl/dioxane (14.5 kg, 55.2 mol, 1.2 eq.) at 5-15° C. was added over 1 h, and the slurry was agitated for 2 h. Ethyl acetate (120 kg) was added. The slurry was agitated for an additional 2 h, and the solids were collected by centrifugation. The solids were dissolved in dichloromethane (350 kg). The solution was washed with 10% aqueous potassium carbonate solution several times, and the solution was dried with sodium sulfate. The filtrate was concentrated to a small volume (˜30 L) under vacuum at 35˜40° C. MTBE (40 kg) and n-heptane (100 kg) were added. The mixture was stirred for 30˜40° C. for 2 h and then concentrated under vacuum at 30˜40° C. to ˜80 L. The solids were collected by centrifugation, and the cake was dried at 35-40° C. for 10 h to give Compound (C), 9.8 kg, 31% yield (over 2 steps), 99.8% pure by HPLC.

### Example 10: Large Scale Production of Compound (C) Using Mg-Generated [1.1.1]Propellane

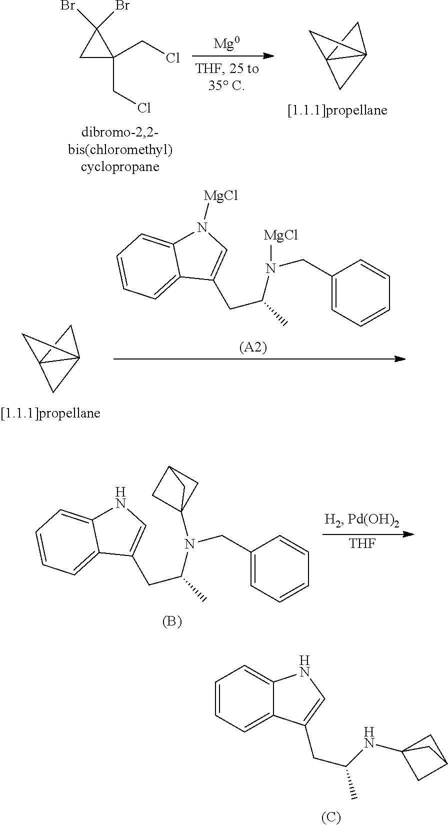

To a mixture of the HCl salt of Compound (A1) (35.0 kg) in DCM (250 kg) in a 1000-L reactor at 10-25° C., aqueous NaOH (129.5 kg, 7.6% wt/wt) was added slowly. The mixture was stirred at 10-25° C. for 2 h. The organic layer was isolated, and the aqueous layer was extracted with DCM (169 kg). The combined organic extractions were washed with aqueous NaOH (100 kg, 5% wt/wt) and brine (58.2 kg, 22% wt/wt), and additional Compound (A1) (29.1 kg—made in a similar fashion as this example) was added, and the combined organic phase was then dried over Na₂SO₄ (20.0 kg). The salts were filtered off, and the cake was washed with DCM (53.4 kg). The filtrate was concentrated under vacuum. THF (168 kg) was added to the residue, and the solvent was removed under vacuum. THF (168 kg) was added to the residue a second time, and the solvent was removed under vacuum. The residue was dissolved in THF (168 kg) to give a solution of Compound (A1) in THF (assay: 20.65%, 58.3 kg Compound A1).

To a mixture of Mg turnings (26.0 kg, 1069.52 mol) in THF (480 kg) in a 2000-L reactor, DIBAL-H (9.1 kg, 1.0 M in hexane) was added. The mixture was stirred at 20±5° C. for 20 min. A portion (˜8%) of the solution of dibromo-2,2-bis(chloromethyl)cyclopropane (132.0 kg) in THF (240 kg) was added slowly, maintaining the internal temperature at <40° C. The remaining solution was then added over 13 h. The mixture was heated to 25-35° C. for 4 h, and then cooled to 0-10° C. to give a [1.1.1]propellane mixture.

The solution of Compound (A1) (58.0 kg based on assay) was added to the above [1.1.1]propellene solution over 30 min, while maintaining the internal temperature at 0-10° C. After 10 min, i-PrMgCl·LiCl (314.0 kg, 1.25 M in THF) was added over 3 h, maintaining the internal temperature at 5-15° C. The reactor was sealed, and the mixture warmed to 25-35° C. for 100 h. The mixture was cooled to 5-15° C. and water (1 kg) was then added. The reactor was sealed and warmed to 25-35° C. for 36 h.

The mixture was cooled to 0-10° C. Cold water (80 kg) was added at 0-10° C. to quench the reaction. The mixture was transferred to a 5000-L reactor, and additional cold water (800 kg) was added at 0-10° C. The mixture was extract with MTBE (202 kg). The aqueous layer was adjusted to pH 7-8 using 20% citric acid (135 kg), and then extracted with MTBE (600 kg). The combined organic extractions were concentrated at 35-40° C. under vacuum to 750-900 L and then diluted with MTBE (550 kg). The solution was washed with 5% citric acid (500 kg), followed by water (500 kg) and 1% aqueous NaOH solution (300 kg). The organic layer was dried over Na₂SO₄ (41 kg). The inorganic salt was filtered off, and the cake was washed with DCM (100 kg). The filtrate was concentrated at 35-40° C. under vacuum. Heptane (250 kg) and DCM (100 kg) were added to the residue. The mixture was concentrated again. The residue was diluted with DCM (40 kg) and heptane (80 kg). The solution was passed through a pad of silica gel (50 kg, 60-100 mesh). The filtrate was concentrated at 40° C. under vacuum. The residue was dissolved in THF (254 kg) to give Compound (B1) solution in THF (yield corrected for purity is 64%).

A solution of Compound (B1) in THF was charged to a 2000-L reactor. The system was purged with N₂ (3×). Pd(OH)₂/C (5.2 kg, 20% wt/wt, wet catalyst) was added. The system was purged with N₂ (3×), and with H₂ (3×). The resulting slurry was agitated at 25-30° C. under 0.06˜0.08 MPa H₂ for 40 h. Additional Pd(OH)₂/C (1.0 kg, 20% wet) was added. The slurry was agitated at 25-30° C. under 0.06˜0.08 MPa H₂ for 40 h. The system was purged with N₂ (3×). The mixture was filtered through a pad of Celite, and the cake was washed with DCM (250 kg). The filtrate was concentrated under vacuum at 35-40° C. to 50˜70 L, which was dissolved in DCM (200 kg). The solution was cooled to 5-15° C. 4 M HCl/dioxane (40.5 kg, 1.1 eq.) was added at 5-15° C. over 1 h. The resulting slurry was agitated for 2 h. EtOAc (267 kg) was added. The slurry was agitated for 2 h, and the solid was collected by centrifugation.

The wet cake was suspended in DCM (595 kg). 10% K₂CO₃ solution (150 kg) was added slowly. After 30 min, the organic layer was isolated, and the aqueous layer was extracted with DCM (100 kg). The combined organic extractions were washed with 10% K₂CO₃ (150 kg). Compound (C) (2.5 kg recovered from last campaign) was added to the solution, which was then passed through a pad of silica gel (60-100 mesh, 50 kg) and washed with MTBE (300 kg). The filtrate was concentrated to 150-200 L under vacuum at 35˜40° C. n-Heptane (220 kg) was added. The mixture was stirred at 30-40° C. for 2 h and then concentrated under vacuum at 30˜40° C. to 150˜200 L. The solid was collected by centrifugation, and the cake was dried at 35-40° C. for 10 h to give Compound (C) (31.8 kg, 55% yield over two steps, 99.8% pure by HPLC).

### Example 11

THF (13.3 kg) was added into a 500 L reactor at 15˜25° C. followed by Compound (E1) (17.8 kg) at 15˜25° C. Additional THF (7.8 kg) was used to rinse solids off the walls of reactor. At 15˜25° C., a solution of NaOH (2.4 kg) in purified water (71.6 kg) was added into the mixture at a rate of 10˜15 kg/h. The mixture was stirred at 15˜25° C. After 18˜20 h, the mixture was cooled to 5˜15° C. At a temperature≤15° C., the pH of the mixture was adjusted to 7.0-8.0 with the addition of a solution of sulfuric acid (3.5 kg) in purified water (71.2 kg). Ethyl acetate (56.2 kg) was added into the mixture and stirred for 0.5˜1.0 h. The temperature of the mixture was adjusted to 5˜15° C. At a temperature≤15° C., the pH of the mixture was adjusted to 6.0˜7.0 with the remaining solution of sulfuric acid (3.5 kg) in purified water (71.2 kg) from the previous pH adjustment step. Finally, at a temperature≤15° C., the pH of the mixture was adjusted to 5.1˜5.4 with a solution of sulfuric acid (1.4 kg) in purified water (53.4 kg). The mixture was stirred for 15˜30 min at a temperature≤15° C., and the phases were allowed to separate. The organic layer was collected. Ethyl acetate (56.1 kg) was added into the aqueous phase at 515° C. The phases were allowed to separate, and the organic layer was collected. Purified water (71.2 kg) was added into the organic phase at 15˜25° C., stirred for 15˜30 min and the phases were allowed to separate. After performing this sequence of washes two more times, the combined organic phase was concentrated at a temperature≤40° C. under reduced pressure until 3˜4 V left. THF in 3 portions (63.2 kg, 63.1 kg, 61.4 kg) was added into the mixture and concentration was done at a temperature≤40° C. under reduced pressure until 3˜4 V left. THF (63.5 kg) was added followed by additional THF (total of 188.7 kg) to ensure residual ethyl acetate≤0.2% and water content ≤0.8%. The mixture was transferred into another 500 L glass-lined reactor through a capsule filter and stirring was initiated. Purified water (4.7 kg) was added, and the mixture was cooled to 5˜15° C. A solution of sulfuric acid (4.1 kg) in acetonitrile (67.4 kg) was added at a rate of 6˜8 kg/h while maintaining the temperature of 5˜15° C. The temperature of the mixture was then adjusted to 15˜25° C. and maintained for 4˜6 h with stirring. The mixture was filtered with a 140 L agitated filter dryer. Acetonitrile (54.5 kg and second charge of 54.1 kg) was used to wash the reactor and transferred to the filter cake. The mixture was then transferred into an agitated Nutsche filter dryer, stirred for 0.5˜1 h and filtered. THF level was above specifications and so additional acetonitrile (54.1 kg+54.2 kg in 2 charges) was added into the mixture with stirring and filtered again until THF level met the specifications. The filter dryer was swept with nitrogen for at least 2 h, and the solid was dried at a temperature≤45° C. for ˜24. The solid was sampled for acetonitrile, THF, ethyl acetate and methanol content. Acetonitrile content was higher than desired and so the solid was sieved through 60 mesh and then the resulting solids were than dried similarly (a temperature≤50° C.) to afford Compound (F) as a H₂SO₄ salt (16.20 kg, 76.6% yield) with a purity with >99%.

### Example 12: Preparation of General Procedure for Preparation of [1.1.1]Propellane

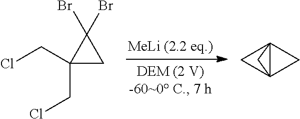 [SMILES: ClCC1(CCl)CC1(Br)Br ; C1C23CC12C3]

An oven dried 5.0 L pressure vessel was cooled with a nitrogen-filled balloon and charged with MeLi (1.37 L, 2.74× by volume) and cooled to −65˜−60° C. A solution of dibromo-2,2-bis(chloromethyl)cyclopropane (500 g, 1.68 mol, 1.00 eq.) in DEM (1.00 L) was added dropwise at −60˜−50° C. After addition, the mixture was allowed to stir for 2 h at −65-60° C. The mixture was warmed to −30° C. and stirred for 4 h at −30° C. The mixture was warmed to 0° C. and stirred for 2 h at 0° C. A solution of N-ethylpiperazine (385 g, 3.37 mol, 2.00 eq.) in DEM (0.5 L) was added dropwise at −5˜0° C. After addition, the mixture was stirred at −5˜0° C. for 12 h. The mixture was distilled under vacuum to afford [1.1.1]propellane solution (4.39 kg, 4.10% by QNMR, 80.8% yield). Overall, 500 g of dibromo-2,2-bis(chloromethyl)cyclopropane was converted to [1.1.1]propellane in 2 batches.

### Example 13: Preparation of General Procedure for Preparation of Compound (B1)

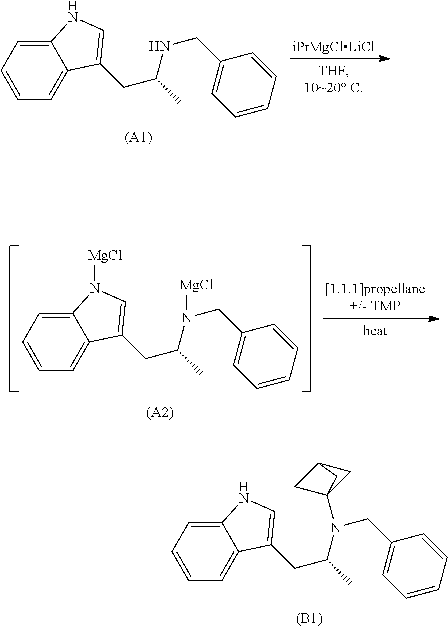

An oven dried 5.0 L pressure vessel was cooled with a nitrogen-filled balloon and charged with Compound (A1) (250 g, 1.00× by weight, KF: 144.2 ppm) and THF (1.75 L, 7.00× by volume, KF: 42.6 ppm). i-PrMgCl·LiCl (1.35 L, 1.75 mol, 1.85 eq.) was added dropwise by at 0-10° C. (internal temperature). The mixture was allowed to stir for 2 h at 0-10° C. Distilled 2,2,6,6-tetramethylpiperidine (TMP) (146.94 g, 1.04 mol, 1.10 eq., KF: 120.1 ppm) was added into the mixture at 0˜10° C. [1.1.1]propellane (2750.4 g, 1.04 mol, 1.10 eq., KF: 262.7 ppm) was then added dropwise into the mixture at 0-10° C. The reaction vessel was sealed and heated at 65-70° C. for 90 h. The mixture was cooled to 0-10° C., and H₂O (4.00 L) was added dropwise. The organic layers were separated, and the aqueous layer was extracted with additional MTBE (4.8 L). The combined organic layers were washed with 20% citric acid solution (0.72 L). The organic layer was further washed with a 5% NaHCO₃ aqueous solution (6 L), dried over Na₂SO₄ and concentrated in vacuum to obtain crude Compound (B1) (270 g, 86.40% yield, 97.27% purity) as a brown oil. ¹HNMR (400 MHz CDCl₃) δ 7.80 (s, 1H), 7.47-7.45 (m, 1H), 7.32-7.30 (m, 2H), 7.26-7.24 (m, 1H), 7.19-7.17 (m, 2H), 7.12-7.10 (m, 2H), 7.10-7.02 (m, 1H), 6.89 (s, 1H), 3.83-3.67 (m, 2H), 3.36-3.32 (m, 1H), 3.03-2.98 (m, 1H), 2.66-2.60 (m, 1H), 2.22 (s, 1H), 1.77-1.69 (m, 6H), 0.99-0.94 (m, 3H).

Surprisingly, it was found that the conditions of Example 10 allow for the facile conversion of Compound (A1) to Compound (B1) under mild temperature conditions. The surprisingly low reaction temperature is beneficial because propellane boils at 35° C. Further, even at the lower temperature, the yield of Compound (B1) exceeded 60%.

TABLE 2

| | Peak Reaction | |
|---|---|---|
| Example | Temperature (° C.) | Yield |
| 1 | 50 | 57% |
| 5 | 50 | 39% |
| 7 | 68 | 80% |
| 8 | 68 | 58% |
| 9 | 55 | 35% |
| 10 | 35 | 64% |
| 13 | 70 | 86% |

For XRPD analysis, PANalytical X' Pert3 X-ray powder diffractometer was used.

### Parameters for XRPD Test

| Parameters | X′ Pert3 |
|---|---|
| X-Ray wavelength | Cu, Kα; |
| | Kα1 (Å): 1.54060 |
| | Ka2 (Å): 1.54443 |
| X-Ray tube setting | 45 kV, 40 mA |
| Scan range (2θ/°) | 3°~40° |
| Step size (2θ/°) | 0.0263° |
| Scan step time (s) | 46.665 |

Compound (C) was recrystallized by (a) Charged Compound (C) (14 g) free base (99.7% HPLC purity; 97.0% ee) to a 50-mL flask, (b) Charged EtOAc (21 mL) to the flask, (c) Warmed the suspension to 75° C. to give a clear solution, (d) Cooled the mixture to ambient temperature over 1 h, (e) Cooled the mixture to 0-5° C. over 30 min, (f) Agitated the slurry at 0-5° C. for 30 min, (g) Collected the solid by filtration and (h) Dried the cake under vacuum at 40° C. for 18 h to give a white solid (10 g, 99.96% pure by HPLC, 99.8% ee, 71% yield).

Furthermore, although the foregoing has been described in some detail by way of illustrations and examples for purposes of clarity and understanding, it will be understood by those of skill in the art that numerous and various modifications can be made without departing from the spirit of the present disclosure. Therefore, it should be clearly understood that the forms disclosed herein are illustrative only and are not intended to limit the scope of the present disclosure, but rather to also cover all modification and alternatives coming with the true scope and spirit of the disclosure provided herein.

What is claimed is:

1. A process of obtaining a compound of Formula (B) comprising: 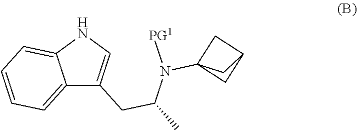
combining a compound of Formula (A), a base and [1.1.1]propellane to afford a compound of Formula (B); wherein the compound of Formula (A) has the structure 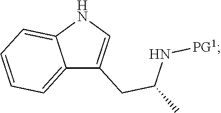
and each PG¹ is a protecting group.

2. The process of claim 1, wherein the reaction conducted at room temperature.

3. The process of claim 1, wherein the reaction is conducted at a temperature in the range of about 25 to about 35° C.

4. The process of any one of claims 1-3, wherein PG¹ is selected from the group consisting of an unsubstituted or a substituted benzyl, a silyl-based protecting group and an unsubstituted allyl.

5. The process of claim 4, wherein PG¹ is an unsubstituted or a substituted benzyl.

6. The process of claim 5, wherein PG¹ is an unsubstituted benzyl.

7. The process of any one of claims 1-6, wherein the base is an organometallic base.

8. The process of claim 7, wherein the organometallic base is an organometallic magnesium base.

9. The process of claim 8, wherein the organometallic magnesium base is a Grignard reagent.

10. The process of claim 7, wherein the organometallic base is an organometallic lithium base.

11. The process of claim 10, wherein the organometallic lithium base is n-butyllithium.

12. The process of claim 7, wherein the organometallic base is an organometallic magnesium-lithium base.

13. The process of claim 12 wherein the organometallic magnesium-lithium organometallic base is (unsubstituted C₁₋₄ alkyl)Mg(halide)-Li(halide).

14. The process of claim 13 wherein (unsubstituted C₁₋₄ alkyl)Mg(halide)-Li(halide) is iPrMgCl·LiCl.

15. The process of any one of claims 1-14, further comprising removing the PG¹ from the compound of Formula (B) to obtain a compound of Formula (C), wherein the compound of Formula (C) has the structure

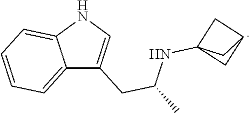 [SMILES: C[C@H](Cc1c[nH]c2ccccc12)NC12CC(C1)C2]

16. The process of claim 15, wherein the PG¹ of the compound of Formula (B) is removed via metal catalyzed hydrogenation or an acid.

17. The process of claim 16, wherein metal catalyzed hydrogenation is palladium catalyzed hydrogenation, platinum catalyzed hydrogenation or nickel catalyzed hydrogenation.

18. The process of claim 17, wherein the catalyst is selected from the group consisting of Pd(OH)₂, Pd/C, Pd(OH)₂/C, silica supported Pd, resin supported Pd, polymer supported Pd, Raney nickel, Urushibara nickel, Ni supported on SiO₂, Ni supported on TiO₂—SiO₂, Pt/C, Pt supported on SiO₂ and Pt supported on TiO₂—SiO₂.

19. The process of claim 16, wherein the PG¹ of the compound of Formula (B) is removed using H₂ and a Pd compound.

20. The process of claim 15, wherein the PG¹ of the compound of Formula (B) is removed using a fluoride source or an acid.

21. The process of claim 20, wherein PG¹ of the compound of Formula (B) is removed using a fluoride source selected from the group consisting of pyridine hydrogen fluoride complex, a triethylamine hydrogen fluoride complex, NaF, tetrabutylammonium fluoride (TBAF) and 1:1 tetrabutylammonium fluoride/AcOH.

22. The process of any one of claims 15-21, further comprising combining the compound of Formula (C) and a compound of Formula (D), optionally in the presence of an acid, to form a compound of Formula (E), wherein the compound of Formula (D) has the structure 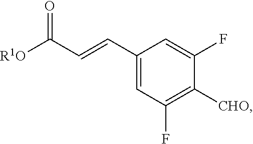
and the compound of Formula (E) has the structure 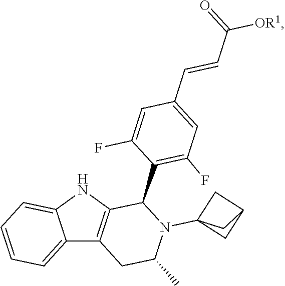
wherein each R¹ is an unsubstituted C₁₋₄ alkyl.

23. The process of claim 22, wherein the acid is acetic acid.

24. The process of any one of claims 22-23, wherein the compound of Formula (C) and the compound of Formula (D) undergo a condensation reaction between the secondary amine of the compound of Formula (C) and the aldehyde of the compound of Formula (D), and then a cyclization reaction to form the compound of Formula (E).

25. The process of any one of claims 22-24, wherein R¹ is methyl.

26. The process of any one of claims 22-25, further comprising hydrolyzing the alkyl ester (—C(═O)OR¹, wherein R¹ is an unsubstituted C₁₋₄ alkyl) of the compound of Formula (E) to a carboxylic acid and afford a compound of Formula (F), wherein the compound of Formula (F) has the structure

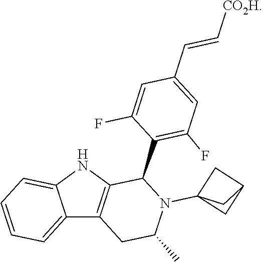 [SMILES: C[C@@H]1Cc2c([nH]c3ccccc23)[C@@H](c2c(F)cc(/C=C/C(=O)O)cc2F)N1C12CC(C1)C2]

27. The process of claim 26, wherein the hydrolysis is conducted using a base.

28. The process of claim 27, wherein the base is selected from the group consisting of NaOH, LiOH and KOH.

29. The process of any one of claims 26-28, further comprising forming the hydrogen sulfate salt of the compound of Formula (F) using a hydrogen sulfate source.

30. The process of claim 27, wherein the hydrogen sulfate source is H₂SO₄.

31. The process of any one of claims 1-30, further preparing the [1.1.1]propellane from dibromo-2,2-bis(chloromethyl)cyclopropane using Mg(0) or an organolithium reagent.

32. The process of claim 31, wherein the organolithium reagent is PhLi or (C₁₋₈ alkyl)Li.

33. The process of any one of claims 1-32, further comprising the use of 2,2,6,6-tetramethylpiperidine in the preparation of the compound of Formula (B).

34. The process of any one of claims 1-33, further comprising the reductive amination of a compound of Formula (1) using an aldehyde and a reducing agent to provide the compound of Formula (A), wherein the compound of Formula (1) has the structure

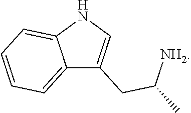

35. The process of claim 34, wherein the reducing agent is selected from the group consisting of sodium borohydride, lithium aluminum hydride, sodium triacetoxyborohydride and sodium cyanoborohydride.

36. The process of claim 34 or 35, wherein the aldehyde is an unsubstituted or a substituted benzylaldehyde or an unsubstituted or a substituted C₁₋₆ alkylaldehyde.

37. The process of claim 36, wherein the aldehyde is an unsubstituted or a substituted benzylaldehyde.

38. A crystalline compound, wherein the compound is crystalline Compound (C).

39. The crystalline compound of claim 38, wherein the crystalline compound is characterized by one or more peaks in an X-ray powder diffraction pattern, wherein the one or more peaks is selected from a peak in the range of from 8.0 to 9.6 °2θ, from 14.8 to 16.4 °2θ, from 16.6 to 18.3 °2θ, from 19.8 to 21.4 °2θ, from 20.5 to 22.1 °2θ and from 23.7 to 25.3 °2θ.

40. The crystalline compound of claim 38, wherein the crystalline compound is characterized by one or more peaks in an X-ray powder diffraction pattern, wherein the one or more peaks is selected from 8.8 °2θ±0.2 °2θ, 15.6 °2θ±0.2 °2θ and 17.4 °2θ±0.2 °2θ.

41. The crystalline compound of claim 38, wherein the crystalline compound is characterized by one or more peaks in an X-ray powder diffraction pattern, wherein the one or more peaks is selected from 20.6 °2θ±0.2 °2θ, 21.3 °2θ±0.2 °2θ and 24.5 °2θ±0.2 °2θ.

42. The crystalline compound of claim 38, wherein the crystalline compound has an X-ray powder diffraction pattern spectrum corresponding to the representative XRPD spectrum depicted in FIG. 1.
